(19) United States
(12) Patent Application Publication
(10) Pub. No.: US 2012/0107764 A1
(43) Pub. Date: May 3, 2012

(54) METHOD FOR PREPARING A SURGICAL MASK FOR THE INSTALLATION OF DENTAL IMPLANTS

(76) Inventor: Rosario RIZZO, Roncadelle (Brescia) (IT)

(21) Appl. No.: 13/284,000

(22) Filed: Oct. 28, 2011

(30) Foreign Application Priority Data
Oct. 29, 2010 (IT) BS2010A000175

(51) Int. Cl. A61C 19/04; A61B 6/14; A61C 9/00

(52) U.S. Cl. 433/44

(57) ABSTRACT

A method for preparing a surgical mask for the installation of a dental implant, including: arranging a pliable material on a support (20) to be positioned in the mouth cavity of a patient to realize the impression (11) of a dental arch and extracting the support with the obtained impression (11); associating a main radiopaque indicator (12) with the impression (11) in the temporary emerging point (α′) for a dental implant to be realized; re-positioning the impression (11) on the dental arch; acquiring at least one radiological image of the patient; elaborating the radiological image so as to obtain processing parameters indicative of the definitive emerging point and the definitive orientation of the dental implant; making a surgical mask to guide an osteotomy operation for a dental implant, the surgical mask being provided with at least one guide hole realized according to the obtained processing parameters.

### CROSS REFERENCE TO RELATED APPLICATIONS

This application claims the benefit of priority under 35 U.S.C. §119 of Italian Patent Application No. BS2010A000175 filed Oct. 29, 2010, the entire contents of which are incorporated herein by reference.

### FIELD OF THE INVENTION

The present invention refers to the field of dental surgery and it concerns particularly a method for preparing a surgical mask intended for aiding the installation of dental implants.

### BACKGROUND OF THE INVENTION

In dental surgery, a missing tooth is often replaced by a prosthetic tooth, being anchored to the maxillary or mandibular bone of a patient by means of a dental implant previously inserted into said bone.

The installation of dental implants needs osteotomy to be carried out, that is the drilling of a bore into the maxillary or mandibular bone to create a seat for the successive implant insertion.

The bore placement and orientation must be determined carefully before the osteotomy could be carried out. As a matter of fact it is necessary to make sure that the bored bony mass is sufficient to sustain the prosthetic device and that the operation does not damage vital bodies. Further, the angle of the definitive prosthetic tooth has to be correct from the physiological point of view and harmonic relatively to the rest of the teeth.

Then to eliminate these parameters the known art uses a method comprising the following steps.

In a first step, the odontologist detects the impression of an incomplete dental arch of the patient, from which a plaster model, or any other suitable material, is obtained.

In a second step, the plaster model is used to cavity form a radiological mask made of radiotransparent material. In the meanwhile, starting from the tooth shape and the masticatory model of the patient, the odontologist determines the point where the osteotomy has to be carried out, that is the ideal emerging point of the dental implant. The position of the desired emerging point is then detected on the radiological mask by means of a radiopaque indicator.

A third step provides for a radiological investigation, usually carried out by means of a computed tomography (TC), of the patient bone structure at the incomplete dental arch.

The investigation is carried out applying a radiological mask in the patient mouth, so that the emerging point the odontologist selected could by detected easily on the radiological image.

In a fourth step, the acquired radiological images are used to detect, by means of appropriate simulation programs, the best orientation of the osteotomy to be made starting from the detected emerging point. In some cases the emerging point could be changed based on bond caused by the patient bone structure.

Starting from ideal parameters detected during the simulation, it is realized therefore a surgical mask, serving for guiding the odontologist drill during the osteotomy operation. The surgical mask in then realized incorporating a surgical template (that is the guide bushing inside of which the drill is introduced during the operation) in the pre-existing radiological mask.

A method for detecting ideal osteotomy parameters and the consequent achievement of a surgical mask according to the known art is disclosed for example in the Published European Application numbered EP 2 046 231.

The methods according to the known art, although supplying substantially the field requirements, have nevertheless some drawbacks.

Particularly, the above proposed method, aimed at guaranteeing a high precision in the determination of the osteosynthesis parameters, asks for at least three sessions for measuring the anatomical patient data: a first session for obtaining the dental arch, a second session for testing the radiological mask and a third session for carrying out the radiological investigations. In fact the times necessary to the accomplishment of the radiological mask are not compatible with the execution in rapid succession of the first and of the third of the above mentioned operations in only one consulting room.

Another important drawback pertains to the absence of a plain and common protocol for the data transmission between various specialists conducting or supervising the different steps of the above described method.

The technical problem the present invention is based on is therefore to devise a method for realizing a personalized radiological mask of the patient in the course of only one session at a skilled consulting room, thereby reducing times and costs to be charged to the patient itself.

### SUMMARY OF THE INVENTION

The afore said technical problem is solved by a method for preparing a surgical mask for the installation of a dental implant, comprising the steps of:

arranging a pliable and auto- or light-hardening material on a support containing radiopaque indicators;

positioning said support in the mouth cavity of a patient so that a dental arch of the patient sinks into said pliable material forming an impression of said dental arch, thanks to the subsequent hardening of the material;

extracting said impression from the mouth cavity of the patient;

optionally associating a radiopaque indicator on said impression at a temporary emerging point for a dental implant that is designed to be inserted into the dental arch;

re-positioning said impression on said dental arch;

acquiring at least one radiological image of the patient including the radiopaque indicators contained in the mask;

elaborating said radiological image so as to obtain processing parameters indicative of the definitive emerging point and the definitive orientation of the dental implant;

making a surgical mask to guide an osteotomy operation in order to achieve a seat for a dental implant, said surgical mask comprising at least one guide hole realized according to the previously obtained processing parameters.

Advantageously, the above outlined method uses directly the impression of dental arch as radiological guide in the successive operations for acquiring the anatomical patient data. In this way, it is possible to acquire all necessary anatomical data in the course of only one session at a skilled consulting room: in fact the patient has only to await the impression solidification on the support containing the radiopaque indicators in order then to submit himself/herself to radiological examinations.

The used pliable material is preferably composed of an autopolymerizing resin. Even if in principle the present method may be realized with any type of paste for dental impressions, the autopolymerizing resins guarantee a higher mechanical strength allowing successive handling of the impression. It has to be noticed that the used pliable material has to guarantee the radiotransparency of the obtained impression.

The radiopaque indicators have preferably a spherical shape and a vitreous structure, particularly advantageous because it does not reflect rays during the radiological examinations thereby assuring a better precision of the acquired images.

The support is preferably made up of a bite fork comprising secondary radiopaque indicators adapted to allow the identification of a resting plane of said bite fork by means of radiologic investigation instruments. Particularly, said radiological indicators are three or more indicators having spherical shape according to the vertices of a polygon.

Such a solution is particularly advantageous because it allows to measure possible support misalignments relatively to the horizontal, returning the processing parameters to an unique reference frame. This contributes to avoid protocol errors in data transmission between different specialists supervising the surgical mask preparation.

The surgical mask is preferably obtained starting from the impression itself, allowing a further saving on preparation time.

Particularly, the step of making a surgical mask may comprise one or more of the following sub-steps:

a sub-step of making the guide hole on the impression by means of a robot, said guide hole being located and directed according to the previously obtained processing parameters (particularly during the execution of such an operation, it is advantageous to position the impression according to a known orientation, blocking the support sustaining the impression by means of a sustaining apparatus);

a sub-step of inserting a surgical template into the guide hole obtained on the impression;

a sub-step of separating the impression from the support after the guide hole has been realized on the impression; and

a sub-step of polishing the impression edges.

It has to be noticed that, if the dental arch of a patient has a plurality of missing teeth, the afore described method may be adapted obviously for preparing a surgical mask comprising a plurality of guide holes intended for the plurality of implants to be installed.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a view of a support used in the method for preparing a surgical mask according to the present invention;

FIG. 2 is a view of a first step of the method for preparing a surgical mask according to the present invention;

FIG. 3 is a view of an intermediate step of the method for preparing a surgical mask according to the present invention;

FIG. 4 is a view of a subsequent intermediate step of the method for preparing a surgical mask according to the present invention;

FIG. 5 is a view of another intermediate step of the method for preparing a surgical mask according to the present invention; and

FIG. 6 is a view of a surgical mask prepared according to the method of the present invention.

### DESCRIPTION OF THE PREFERRED EMBODIMENTS

Referring to enclosed figures, and particularly to FIG. 6, we identify with numeral 1 a surgical mask intended for aiding the installation of a dental implant at an incomplete dental arch 100 of a patient.

Such a surgical mask 1 includes an impression 11 of the incomplete dental arch 100. The impression has a guide through-hole 13, inside which a surgical template 14 is accommodated, made of titanium or in acetalic resin.

During the osteotomy operation, thanks to which the odontologist will realize the seat for the dental implant to be installed, the surgical mask is associated with the dental arch 100. The odontologist then realizes the seat inserting the drill in the surgical template 14, serving as instrument guide.

As mentioned in the previous description of the known art, the placement and the orientation of the seat obtained by means of osteotomy have a critical significance in determination of aesthetic and structural outcome of the prosthetic reconstruction. In the present treatise, the resting position of such a seat is detected by means of an emerging point α (the arch point on which the seat is opened) and an orientation x, that we will define as definitive to distinguish them from temporary parameters used in the intermediate decision-making steps of the method according to the present invention. To simplify the treatise, we will define the resting position of the guide hole 13 present on the surgical guide with the same parameters, even if such a guide hole 13 coincides really with the prosthetic seat to be realized only when the surgical mask 1 is applied to the dental arch 100.

To arrange the surgical mask 1 defining the definitive emerging point α and orientation x optimally, the method according to the invention includes the herein after described steps.

Firstly, the impression 11 of the surgical mask 1 is realized.

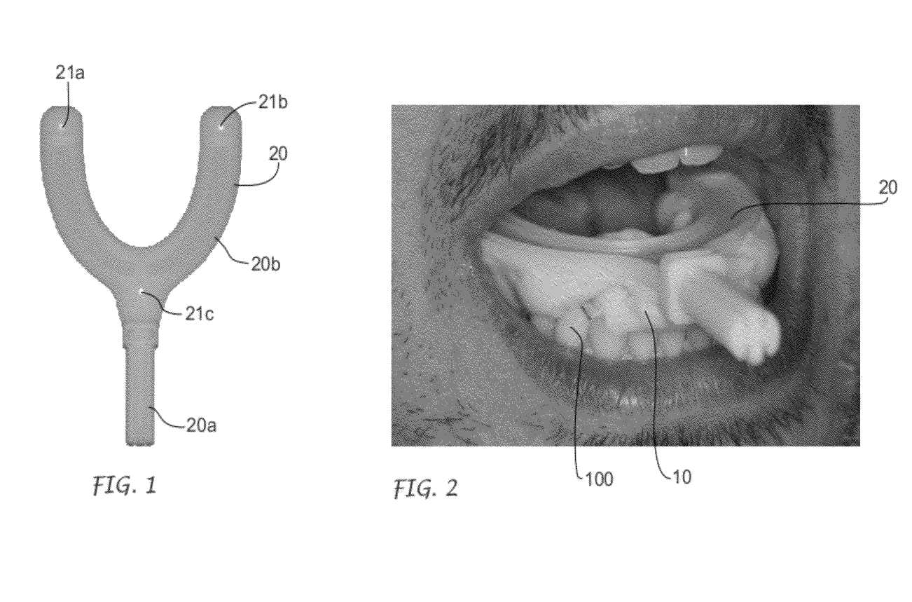

To this purpose firstly a support 20 is arranged, visible in the appended FIG. 1, that in the present embodiment does materialize in a bite fork 20 including a main body 20b, shaped as horseshoe to reproduce the shape of a dental arch, and a grip 20a that it is engaged on said main body 20b to allow the introduction thereof inside of the mouth cavity of the patient.

Advantageously, said bite fork 20 is made of radiotransparent material and it comprises three secondary radiopaque indicators 21a, 21b, 21c of spherical shape placed at the two free ends of the main body 20b and at the engaging point of the grip 20a on the main body 20a. The indicators, preferably made of vitreous material, are then disposed on the resting plane of the bite fork 20 based on the triangle vertices, and thereby allowing such a plane to be identified on a radiological image.

On one of the faces of the main body 20b of the bite fork 20 a pliable material is arranged, that in the case in point is composed of an autopolymerizing resin.

By handling the grip 20a, the bite fork 20 is positioned into the mouth cavity of the patient in such a way that the dental arch 100 sinks into the autopolymerizing resin 10, achieving a mold determining the desired impression 11. Such an operation is illustrated in FIG. 2.

The bite fork 20 having the impression 11 is then drawn out from the mouth cavity of the patient.

It has to be noticed that the autopolymerizing resin 10 cures after the impression has been realized, losing the before detected compliance abilities.

Analyzing the achieved impression 11, it is decided therefore in which point of the dental arch 100 the dental implant is designed to be inserted, determining a temporary emerging point α′ the position thereof is indicated on the impression 11 itself by means of a main radiopaque indicator 12.

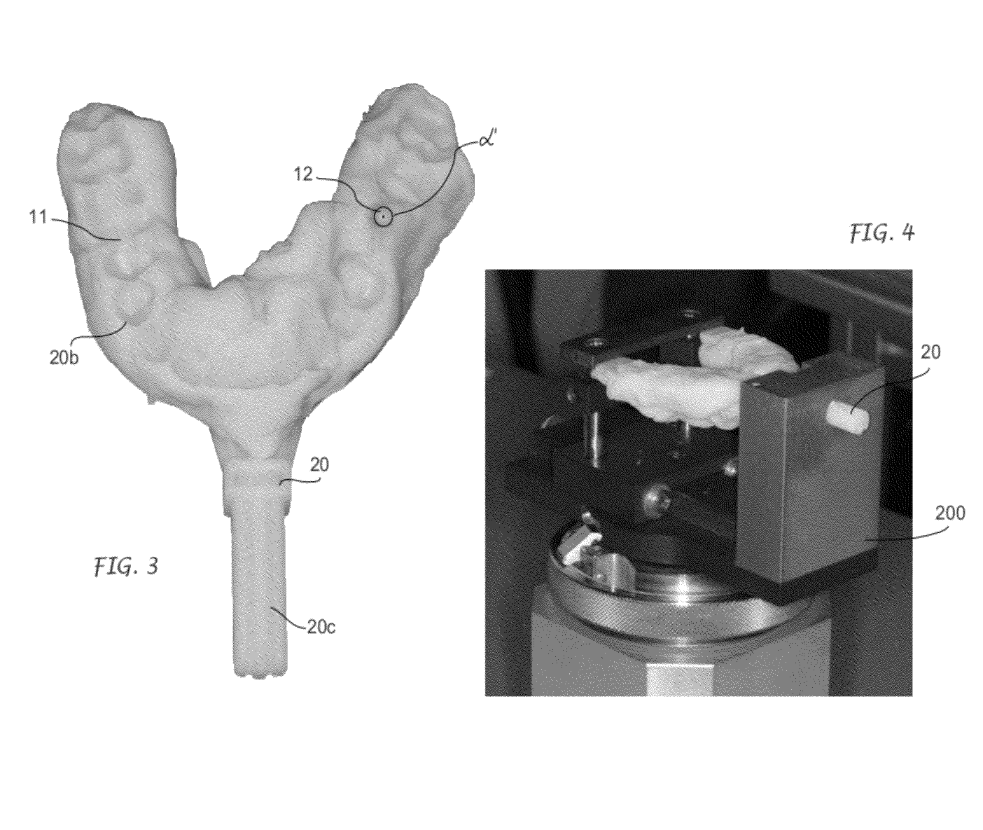

Such a main radiopaque indicator 12, as can be seen in FIG. 3, is composed of a sphere made of vitreous material, that is associated with the impression integrating it into the autopolymerizing resin 10 preferably before its complete curing.

Then the impression 11, associated with the main radiopaque indicator 12, carried by the bite fork 20, is placed again on the dental arch 100 of the patient to act as a radiological mask.

The patient is then subjected to radiological investigation, particularly to a computed tomography (TC) to acquire at least one radiological image of the patient mouth. Preferably the patient is subjected to a plurality of computed tomographies carried out according to overlapped horizontal planes.

In this step it is important to make sure that the acquired images show the whole radiopaque indicators carried by the bite fork 20, that is the three secondary indicators 21a, 21b, 21c defining the resting position and the main indicator 12 detecting the temporary emerging point α′ for the dental implant.

The acquired radiological images are then sent to an appropriate managing program, allowing to plan virtually the resting position of the seat of the dental implant, verifying the latter is compatible with the patient bone structure.

By means of the managing program the ideal value is then searched for obtaining the dental implant starting from the temporary emerging point α′ identified by the main radiopaque indicator 12. If no orientation is assessed satisfactory, it goes on modifying the emerging point, until the definitive emerging point α and the definitive orientation x are detected.

The program then calculates the processing parameters indicative of the definitive emerging point α and the definitive orientation.

It has to be noticed that from the position of the three secondary radiopaque indicators 21a, 21b, 21c, the program is able to assess the inclination of the bite fork 20 relatively to the horizontal. Based on this, the program will correct the processing parameters adapting them to a reference in which the bite fork 20 lies on a horizontal plane.

The subsequent step of the method concerns the proper achievement of the surgical mask 1 starting from the previously obtained processing parameters.

The surgical mask 1 is obtained starting from the previously obtained impression 11, that is drilled by an anthropomorphous robot to realize the guide hole 13 where the surgical template 14 is accommodated.

Firstly the bite fork 20 is located on a horizontal plane, blocking the ends thereof to a reference sustaining apparatus 200 for an anthropomorphous robot, as illustrated in FIG. 4. In this step the main radiopaque indicator 12 is taken away from the impression 11 to not impede the drilling operations.

The anthropomorphous robot will carried out the guide hole 13 by means of a milling device assembled on its mobile arm, based on the previously determined processing parameters.

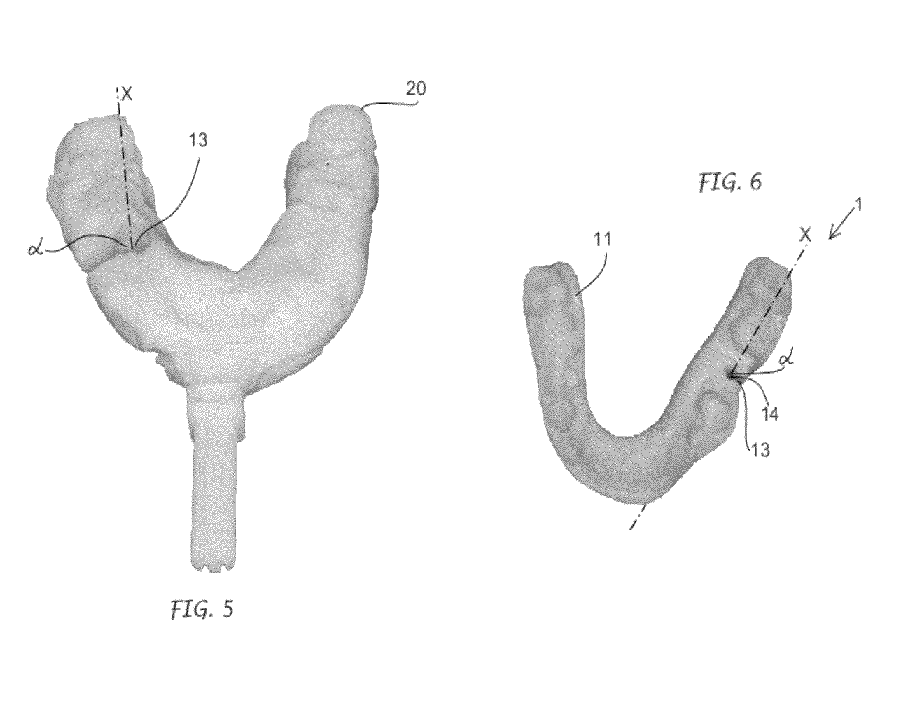

The drilled impression 11, sustained by the bite fork 20, is illustrated in the appended FIG. 5.

Finally, the bite fork 20 is drawn out from the sustaining apparatus 200 and the impression 11 is refinished to obtain the definitive surgical mask 1 visible in FIG. 6. The refinishing operations comprise the detachment of the bite fork 20 from the impression 11, the introduction of the surgical template 14 into the guide hole 13 and the polishing of the edges of the impression 11.

The so achieved and refinished surgical mask 1 then may be located on the dental arch 100 of the patient to guide the osteotomy operation designed to obtain the dental implant seat to be installed.

Obviously a person skilled of the art may bring various changes and variations to the method afore described, to satisfy contingent and specific requirements.

While specific embodiments of the invention have been shown and described in detail to illustrate the application of the principles of the invention, it will be understood that the invention may be embodied otherwise without departing from such principles.

1. A method for preparing a surgical mask for the installation of a dental implant, comprising the steps of:
arranging a pliable material on a support;
positioning said support in the mouth cavity of a patient so that a dental arch of the patient sinks into said pliable material forming an impression of said dental arch;
extracting said impression from the mouth cavity of the patient;
associating a main radiopaque indicator on said impression at a temporary emerging point for a dental implant that is designed to be inserted into the dental arch;
re-positioning said impression on said dental arch;
acquiring at least one radiological image of the patient including the main radiopaque indicator associated with the impression;
elaborating said radiological image so as to obtain processing parameters indicative of the definitive emerging point and definitive orientation of the dental implant;
making a surgical mask to guide an osteotomy operation in order to achieve a seat for a dental implant, said surgical mask comprising at least one guide hole realized according to the previously obtained processing parameters.

2. A method for preparing a surgical mask according to claim 1, wherein said pliable material is made of an autopolymerizing resin.

3. A method for preparing a surgical mask according to claim 1, wherein said main radiopaque indicator has a spherical shape.

4. A method for preparing a surgical mask according to claim 1, wherein said support is made up of a bite fork comprising secondary radiopaque indicators adapted to allow the identification of a resting plane of said bite fork by means of radiologic investigation instruments.

5. A method for preparing a surgical mask according to claim 4, wherein said secondary radiopaque indicators are three round shaped indicators positioned according to the vertices of a triangle.

6. A method for preparing a surgical mask according to claim 1, wherein said surgical mask is made starting from the impression.

7. A method for preparing a surgical mask according to claim 6, wherein the step of making the surgical mask comprises a sub-step of making the guide hole on the impression by means of a robot, said guide hole being located and directed according to the obtained working parameters.

8. A method for preparing a surgical mask according to claim 7, wherein the step of making the surgical mask comprises a sub-step of inserting a surgical template into the guide hole obtained on the impression.

9. A method for preparing a surgical mask according to claim 7, wherein during the sub-step of making a guide hole on the impression, said impression is positioned according to a known orientation by blocking the support supporting it on a sustaining apparatus.

10. A method for preparing a surgical mask according to claim 7, wherein the step of making the surgical mask comprises a sub-step of separating the impression from the support after the guide hole has been realized on the impression.

11. A method for preparing a surgical mask according to claim 8, wherein during the sub-step of making a guide hole on the impression, said impression is positioned according to a known orientation by blocking the support supporting it on a sustaining apparatus.

12. A method for preparing a surgical mask according to claim 8, wherein the step of making the surgical mask comprises a sub-step of separating the impression from the support after the guide hole has been realized on the impression.

13. A method for preparing a surgical mask according to claim 9, wherein the step of making the surgical mask comprises a sub-step of separating the impression from the support after the guide hole has been realized on the impression.
